(19) United States
(12) Patent Application Publication
(10) Pub. No.: US 2015/0005673 A1
(43) Pub. Date: Jan. 1, 2015

(54) DETERMINATION OF THE MOTION OF AN EXAMINATION REGION

(71) Applicant: Siemens Aktiengesellschaft, Munich (DE)

(72) Inventors: Thomas ALLMENDINGER, Forchheim (DE); Thilo HANNEMANN, Erlangen (DE); Andre HENNING, Erlangen (DE); Javier PENA, Nuernberg (DE); Florian PFANNER, Uttenreuth (DE)

(73) Assignee: SIEMENS AKTIENGESELLSCHAFT, Munich (DE)

(21) Appl. No.: 14/294,521

(22) Filed: Jun. 3, 2014

(30) Foreign Application Priority Data
Jul. 1, 2013 (DE) 102013212820.7

(51) Int. Cl. A61B 5/113; A61B 5/00

(52) U.S. Cl. CPC A61B 5/113; A61B 5/004
USPC 600/595

(57) ABSTRACT

A method is used to determine motion of an examination region of a patient. It is based on the use of a flat antenna arrangement, including at least one transmit unit and several receive units, as well as on the use of the transmit unit actuated by a control signal for the transmission of radar signals in the direction of the examination region, and the use of receive units for the receipt of radar signals reflected by the examination region. The method further includes the read-out of receive signals from the receive units, with the receive signals corresponding to the radar signals received. The assignment of the radar signals received to the transmit unit which transmitted the radar signals received in each case, by correlating the receive signals to the control signal, advantageously makes possible the adjustment of parameters derived from the correlated receive signals to retrievably stored model data.

### PRIORITY STATEMENT

The present application hereby claims priority under 35 U.S.C. §119 to German patent application number DE 102013212820.7 filed Jul. 1, 2013, the entire contents of which are hereby incorporated herein by reference.

### FIELD

At least one embodiment of the invention generally relates to a method and a medical diagnostic or therapeutic device for determining the motion of an examination region.

### BACKGROUND

For many medical examinations and treatments it is advantageous to determine motions of a patient, such as the heartbeat or respiratory motion for example. In particular in examinations or treatments using imaging modalities such as computed tomography or magnetic resonance tomography it may be important to determine the motion of a patient. Furthermore, the determination of the motion of a patient may also be important for therapeutic treatment using a radiotherapy device. The motion recorded may be used for motion correction of the image data obtained or for triggering. Often the motion data provides information about physiological parameters such as the heart rate or respiratory rate. In order to determine such motions or physiological parameters, the use for example of an ECG to determine the heart rate, and the use of a respiratory belt to determine the respiratory rate, are known.

A method for sensing information about the position and/or motions of the body of a living organism or of part of the inside of a body is known from DE 102 59 522 A1. The method comprises the transmission of an electromagnetic signal to a predefined region of the body of the living organism, and the receipt of an electromagnetic signal reflected from the region of the body, and the evaluation of the receive signal received in respect of the difference between propagation time and/or frequency and the transmit signal. The method is used to ascertain the information and is characterized in that frequencies in the high-frequency range, in particular in the radar range, are used.

### SUMMARY

At least one embodiment of the invention specifies a method to determine the motion of an examination region of a patient.

At least one embodiment of the invention is directed to an apparatus and/or a method. Features, advantages or alternative embodiments mentioned in the process can also be applied to the other objects and vice versa. In other words the claims (which are directed toward an apparatus for example) can also be developed with the features described or claimed in connection with a method. The corresponding functional features of the method are hereby formed by corresponding objective modules.

At least one embodiment of the inventive method is used to determine the motion of an examination region of a patient. It is based on the use of a flat antenna arrangement, comprising at least one transmit unit and several receive units, as well as on the use of the transmit unit actuated by a control signal to transmit radar signals in the direction of the examination region, and the use of receive units to receive radar signals reflected by the examination region. At least one embodiment of the method further comprises the read-out of receive signals from the receive units, with the receive signals corresponding to the radar signals received. The inventors have recognized that the assignment of the radar signals received to the transmit unit which transmitted the radar signals received in each case, by correlating the receive signals to the control signal, advantageously makes it possible to adjust parameters derived from the correlated receive signals to retrievably stored model data. In this case the model data relates to the motion of the examination region, so that the motion of the examination region can be precisely determined.

According to another aspect of at least one embodiment of the invention the medical diagnostic or therapeutic device can be designed to carry out at least one embodiment of the inventive method.

According to another aspect the medical diagnostic or therapeutic device comprises a patient table, into which the flat antenna arrangement is integrated. The integration means the antenna arrangement is embodied to be very space-saving, and the inventive method can be carried out particularly simply, in particular without attaching a belt or other measurement systems to the patient.

### BRIEF DESCRIPTION OF THE DRAWINGS

The invention is described and explained in more detail below with reference to the example embodiments illustrated in the figures, in which:

FIG. 1 shows a plan view of an embodiment of an inventive antenna arrangement,

FIG. 2 shows a side view of an embodiment of an inventive antenna arrangement,

FIG. 3 shows a circuit diagram of an embodiment of an inventive radar system,

FIG. 4 shows an embodiment of an inventive computed tomography system,

FIG. 5 shows the I and Q components of a regular respiratory motion,

FIG. 6 shows the I and Q components of two overlaid motions, and

FIG. 7 shows a flow chart of an embodiment of the inventive method.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

At least one embodiment of the inventive method is used to determine the motion of an examination region of a patient. It is based on the use of a flat antenna arrangement, comprising at least one transmit unit and several receive units, as well as on the use of the transmit unit actuated by a control signal to transmit radar signals in the direction of the examination region, and the use of receive units to receive radar signals reflected by the examination region. At least one embodiment of the method further comprises the read-out of receive signals from the receive units, with the receive signals corresponding to the radar signals received. The inventors have recognized that the assignment of the radar signals received to the transmit unit which transmitted the radar signals received in each case, by correlating the receive signals to the control signal, advantageously makes it possible to adjust parameters derived from the correlated receive signals to retrievably stored model data. In this case the model data relates to the motion of the examination region, so that the motion of the examination region can be precisely determined.

Because of the plurality of receive units, which are located in a flat antenna arrangement, the assignment of the radar signals received produces additional spatial information regarding the propagation of the radar signals. This additional spatial information results in an improved adjustment of at least one embodiment of the inventive parameters to model data. For this, the model data must of course take the additional spatial information itself into account.

The parameters are adjusted, in accordance with at least one embodiment of an aspect of the invention, to model data which relates to the change over time of the volume of the examination region. As a result, the inventively increased information content can be used to determine a particular, especially important aspect of the motion of the examination region. The change in the volume of organs such as, for example, the lungs or the heart enable conclusions to be drawn regarding malfunctions and illnesses.

According to another aspect of at least one embodiment of the invention, the adjustment is made at least to model data which relates to the frequency spectrum of the motion of the examination region. In this way the regularity of the motion can be recorded, which is important for identifying arrhythmic motions of the heart, for example.

According to another aspect of at least one embodiment of the invention, the examination region relates to the patient's lungs, with radar signals being transmitted and received at a scan rate of at least 10 Hz. This is generally the minimum frequency needed to be able to determine the frequency of the motion of the lungs reliably.

According to another aspect of at least one embodiment of the invention the adjustment comprises both the adjustment to model data for thoracic respiration and the adjustment to model data for abdominal respiration. Valuable information for diagnosis and/or for controlling further devices can be obtained from the additional differentiation between thoracic respiration and abdominal respiration.

According to another aspect of at least one embodiment of the invention, the examination region relates to the patient's heart, with radar signals being transmitted and received at a scan rate of at least 500 Hz. This is generally the minimum frequency needed to be able to determine the frequency of the motion of the heart reliably.

According to another aspect of at least one embodiment of the invention, the model data is based on a trained model of the examination region. The adjustment to model data, based on a trained model, takes place—with appropriately good training—very precisely and permits the motion of the examination region to be determined exactly.

In another embodiment of the invention this comprises the post-processing of image data of the examination region recorded during receipt and transmission, using the adjusted parameters. As a result, artifacts relating to the motion of the examination region can be corrected.

According to another aspect of at least one embodiment of the invention the transmit units and receive units are located in the immediate vicinity of the patient, so that principally the near-field portion of the transmitted radar signals is reflected and received. As a result the antenna arrangement is particularly simple and space-saving to manage.

Another embodiment of the invention comprises the control of a medical diagnostic or therapeutic device using the adjusted parameters. As a result the precision of each controlled function of the medical diagnostic or therapeutic device can be increased, which ultimately results in a better diagnosis or therapy.

According to another aspect of at least one embodiment of the invention the medical diagnostic or therapeutic device can be designed to carry out at least one embodiment of the inventive method.

According to another aspect the medical diagnostic or therapeutic device comprises a patient table, into which the flat antenna arrangement is integrated. The integration means the antenna arrangement is embodied to be very space-saving, and the inventive method can be carried out particularly simply, in particular without attaching a belt or other measurement systems to the patient.

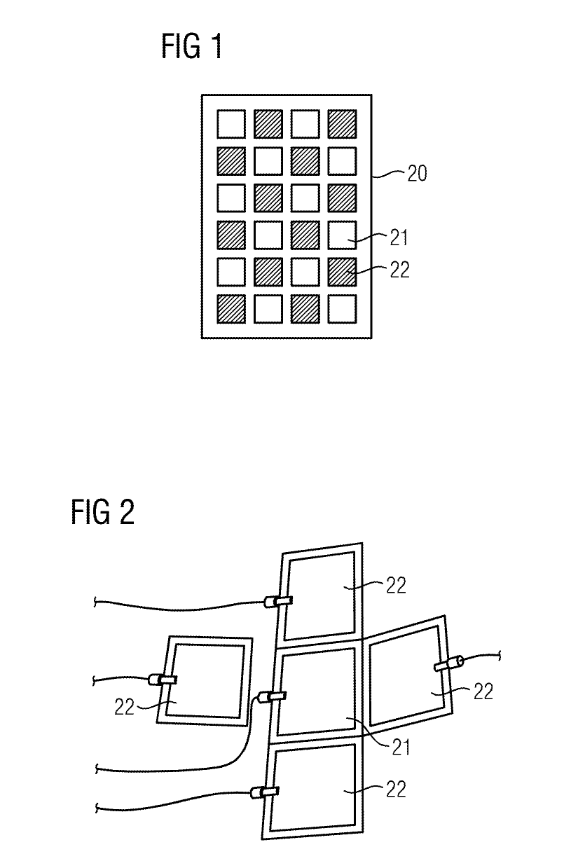

FIG. 1 shows a plan view of an embodiment of an inventive antenna arrangement which is particularly suitable for carrying out an embodiment of the inventive method. The antenna arrangement 20 is embodied to be flat and comprises individually actuatable transmit units 21 for the transmission S of radar signals and individually readable receive units 22 for the receipt E of radar signals. In the example shown here the transmit units 21 are shown in white and the receive units 22 are hatched. The antennas of the transmit units 21 and of the receive units 22 are each embodied in the form of patch antennas. A patch antenna is a flat, often rectangular antenna, whose edge length can in particular have a value of λ/2, where λ is the wavelength at which the antenna acts as a resonator.

An embodiment of the inventive antenna arrangement 20 can be embodied such that both transmit units 21 and receive units 22 are designed for the transmission S and receipt E of radar signals. In other words, in certain embodiments of the invention transmit units 21 can act as receive units 22 (and vice versa). An embodiment of the inventive antenna arrangement 20 can however be embodied such that the transmit units 21 are provided only for the transmission S of radar signals and the receive units 22 only for the receipt E of radar signals. In the latter case, the transmit units 21 and the receive units 22 can, as shown here, be arranged in a chessboard pattern; they can however also form other patterns, in so far as this makes technical sense.

In plan view in the example of the transmit units 20 and receive units 22 shown in FIG. 1 only the antennas are visible in each case. The antennas are embodied identically in the example shown here. The antennas of the transmit units 21 and of the receive units 22 can however also be shaped differently or be otherwise differently embodied, in order to improve the transmit properties or the receive properties. The antennas shown here can have different edge lengths, typically in the region of several centimeters. In particular, resonances at 915 MHz, 868 MHz and 433 MHz are desired, which corresponds to edge lengths of approx. 16.4 cm, 17.3 cm and 34.6 cm in patch antennas. The antennas visible in FIG. 1 have edge lengths of approx. 10 cm to 50 cm, so that the antenna arrangement 20 has dimensions of approx. 0.5 m to 1.5 m wide and 1 m to 2 m long. Both the individual antennas and the entire antenna arrangement 20 can have dimensions and shapes differing from the embodiments cited here by way of example, in so far as this makes technical sense.

FIG. 2 shows a plan view of an embodiment of the an inventive antenna arrangement which is particularly suitable for carrying out the inventive method. In the embodiment shown here the antenna arrangement 20 merely has one centrally arranged transmit unit 21 for the transmission S of radar signals. Furthermore, the antenna arrangement 20 has four receive units 22 arranged symmetrically around the transmit unit 21, so that the entire antenna arrangement 20 is cruciform. The transmit and receive units each have a patch antenna which each have edge lengths of approx. 10 cm to 50 cm. The cables shown in FIG. 2 produce a connection between the antenna unit 20 and a control and evaluation unit 19 for the purpose of data transmission, with the control and evaluation unit 19 being designed to actuate the transmit units 21 via local oscillators 12 and to evaluate the receive signals of the receive units 22.

In the embodiment shown here the individual receive units 22 are connected together by hinges, so that the respective angles between the transmit unit 21 and the receive units 22 can be adjusted. As a result, the antenna arrangement 20 to a certain extent matches the contour of a patient 3 even if the antennas or transmit and receive units are embodied to be fixed, if the antenna arrangement 20 is placed directly on the patient 3, in particularly directly on or under his body. The hinges of the connection of the transmit or receive units can also be embodied as a click connection, so that the number of transmit or receive units in an antenna arrangement 20 can be varied. The embodiment of the antenna arrangement 20 shown here is in particular suitable for determining the motion on the basis of the respiration of the patient 3, by being placed on or under the thorax and/or abdomen of a patient 3.

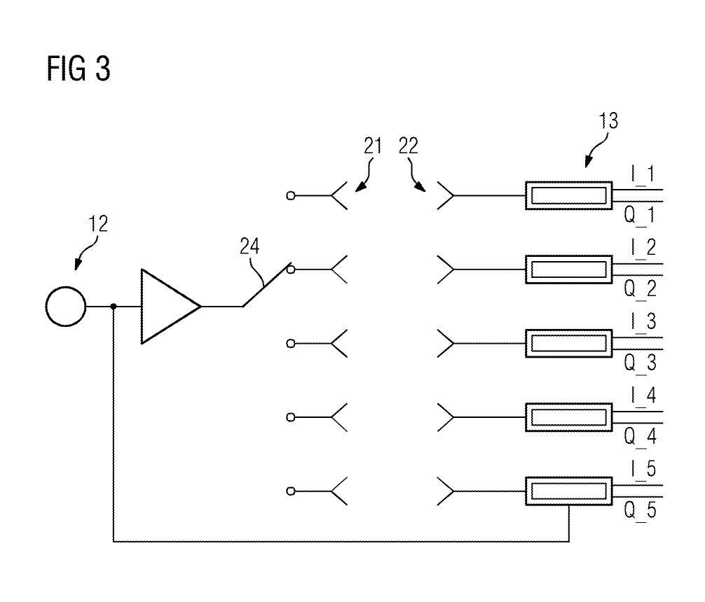

FIG. 3 shows a circuit diagram of the inventive radar system. The local oscillator 12 generates a signal frequency, typically in the range between 100 MHz and 5 GHz. The signal generated by the local oscillator is amplified to the desired transmission power by the power amplifier shown as a triangle. In the example shown here the signal is transmitted by the switch 24 consecutively to the transmit units 21, with each of the transmit units 21 having an antenna for the transmission S of a radar signal with the signal frequency. The radar signals transmitted by a transmit unit 21 can be received by the receive units 22, with each of the receive units 22 comprising an antenna in the example shown here. The receive signals are demodulated by the I/Q demodulators 13 and in each case are converted into an I component (I_{—}1 to I_{—}5) and in each case into a Q component (Q_{—}1 to Q_{—}5). In this case a receive signal is split such that a part is demodulated with the original phase position and produces the I component, with the second part being demodulated phase-shifted by 90° and producing the Q component.

In the example shown here the I/Q demodulator 13 is operated with the same signal frequency as the transmit units 21. In another embodiment, not shown here, the I/Q demodulators 13 are operated with an intermediate frequency which differs slightly, typically in the region of a few kHz, from the signal frequency. Furthermore, the number of transmit units 21 and receive units 22 used can of course vary, in particular the number of transmit units 21 and of receive units 22 in an inventive radar system can differ. Other electronic components such as mixers, filters, amplifiers, etc. can also be used to generate the desired control signal or to demodulate and further process the receive signal, in particular to enable an embodiment of an inventive assignment Z. In another embodiment, the demodulation takes place digitally.

In the embodiment shown here the transmit units 21 do not transmit their respective radar signals simultaneously. Instead the transmit units 21 transmit a temporal series of radar signals, with the transmit units 21 being located at different spatial positions. Thus the transmit units 21 transmit a temporal series which uses the instant of transmission (or receipt) to enable conclusions to be drawn about the spatial position of the transmit unit 21 which transmitted the respective radar signal. However, because of the very small time delay when a radar signal is reflected by a patient 3, the absolute instant of the transmission S of a radar signal is not compared to the receipt E of the radar signal. Instead, conclusions are drawn about the spatial position of the transmit unit 21 which transmitted the radar signal received by correlating the control signal which corresponds to the radar signal transmitted with the receive signal which corresponds to the radar signal received.

It is known in principle from the field of radar technology to draw conclusions about the motion and/or position of an object by correlating a control signal and a receive signal, in particular with the help of an I/Q demodulator. However, it is not known for the information content obtained for medical use using a radar system to be increased by correlating control signals and receive signals. This is particularly the case because the I/Q demodulation can be carried out not only for a permanently assigned pair of antennas, but in principle for the combination of each transmit unit 21 with each receive unit 22. In the embodiment shown here all transmit units 22 can simultaneously receive the radar signals transmitted by a transmit unit 21.

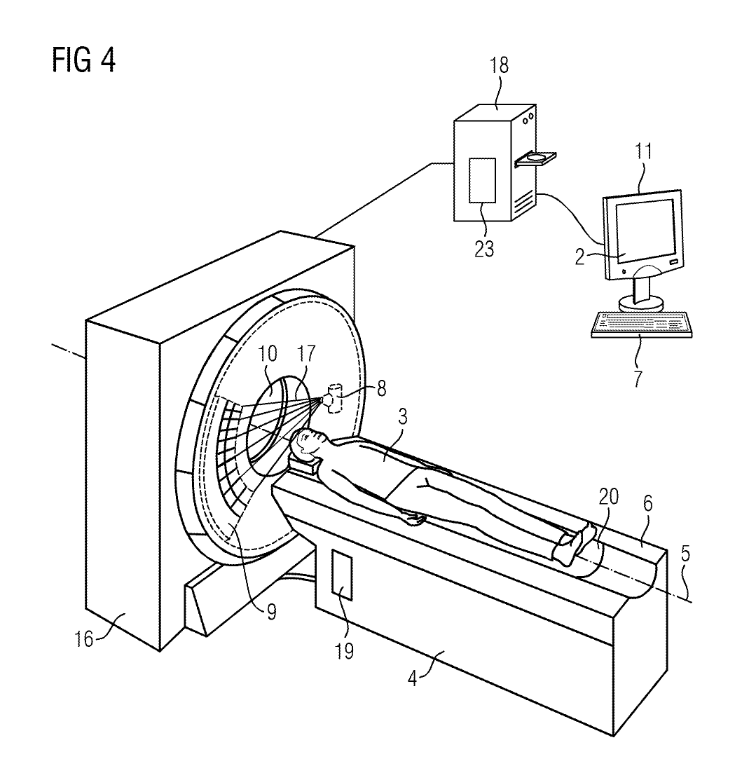

FIG. 4 shows an embodiment of an inventive computed tomography system. The computed tomography system relates to an example embodiment of a medical diagnostic or therapeutic device. The computed tomography system shown here has a recording unit, comprising an X-ray source 8 and an X-ray detector 9. The recording unit rotates about a longitudinal axis 5 during the recording of a tomographic image, and the X-ray source 8 emits X-rays 17 during the spiral recording. While an image is being recorded the patient 3 lies on a patient table 6. The patient table 6 is connected to a table base 4 such that it supports the patient table 6 bearing the patient 3. The patient table 6 is designed to move the patient 3 along a recording direction through the opening 10 of the gantry 16 of the computed tomography system. In the example shown here the antenna arrangement 20 of the inventive radar system is integrated into the patient table 6.

In the present example embodiment the invention comprises a control and evaluation unit 19 which is integrated into the table base 4 and accordingly is always located outside the beam path of the X-rays 17. The control and evaluation unit 19 can additionally, in a manner not shown, be shielded against scattered X-rays, for example with a plate or a housing made of lead. The control and evaluation unit 19 is also connected to the computer 18 to exchange data. The control and evaluation unit 19 can in particular comprise one or more local oscillators 12 and one or more I/Q demodulators 13. In particular, if the antenna arrangement 20 is embodied as a flexible mat which can be placed on the patient 3, the control and evaluation unit 19 can also be accommodated in a separate housing outside the patient table 6 or the table base 4. In each case it is advantageous to protect the control and evaluation unit 19 against X-rays by a corresponding sheathing.

It is the function of the control and evaluation unit 19 to actuate the antenna arrangement 20 and thus the individual transmit units 21 using a control signal and to read out receive signals from the individual receive units 22. The control signal can in particular be generated by at least one local oscillator 12 and if appropriate by further electronic components such as a mixer, amplifier or filter. The control and evaluation unit 19 shown here is designed for the assignment Z of a radar signal received by a receive unit 22 to the transmit unit 21 which transmitted the radar signal received, by correlating the control signal with the receive signal. The control and evaluation unit 19 is furthermore designed to receive signals from a computer 18 or to transmit signals to the computer 18.

In the example shown here the medical diagnostic or therapeutic unit is designed in the form of a computed tomography system by a determination unit 23 in the form of a stored computer program that can be executed on a computer 18, to determine the motion of an examination region of a patient 3. It is generally the case that the determination unit 23 can be embodied in the form of both hardware and software. For example, the determination unit 23 can be embodied as a so-called FPGA (acronym for “Field Programmable Gate Array”) or can comprise an arithmetic logic unit. Other than shown here, the determination unit 23 can also be located in the immediate vicinity of the control and evaluation unit 19 or can be embodied together therewith as a compact unit. In particular the determination unit 23 can also be integrated into the table base 4.

Furthermore, in the example shown here the medical diagnostic or therapeutic unit is designed to use the motion determined by an embodiment of the inventive radar system for the control St of the medical diagnostic or therapeutic unit and/or for the post-processing Nb of data obtained by the medical diagnostic or therapeutic unit. The data can for example be image data. The medical diagnostic or therapeutic unit can be designed for the control St and the post-processing Nb in particular by a computer program retrievably stored on the computer 18. The control St comprises the irradiation of the patient 3, for example with electromagnetic radiation, electrons or particles, depending on the type of the medical diagnostic or therapeutic unit. Thus the irradiation may for example take place only in the resting phase of the heart of the patient 3 or a particular position of the thorax of the patient 3 which depends on the respiratory motion. The intensity of the radiation or the angle of radiation can also be adjusted by control St. In another embodiment the control St comprises positioning the patient 3 by moving the patient table 6. The post-processing Nb relates for example to the segmentation or registration of a temporal series of images, based on image data, of a moving examination region.

The computer 18 is connected to an output unit 11 and an input unit 7. The output unit 11 is for example one (or more) LCD, plasma or OLED screen(s). The output 2 on the output unit 11 comprises for example a graphical user interface for actuating the individual units of the computed tomography system and the control and actuation unit 19. Furthermore, different views of the recorded data can be displayed on the output unit 7. The input unit 7 is for example a keyboard, mouse, so-called touch screen or even a microphone for speech input.

In other example embodiments, not shown here, the medical diagnostic or therapeutic device may relate to imaging devices other than a computed tomography system, for example a magnetic resonance tomography system or a C-arm X-ray device. The medical diagnostic or therapeutic device may furthermore be designed to use positron emission tomography. Furthermore, the medical diagnostic or therapeutic device may relate to a device which is designed to emit electromagnetic radiation and/or electrons and/or particles such as ions for example and thus is suitable for use in radiotherapy or particle therapy.

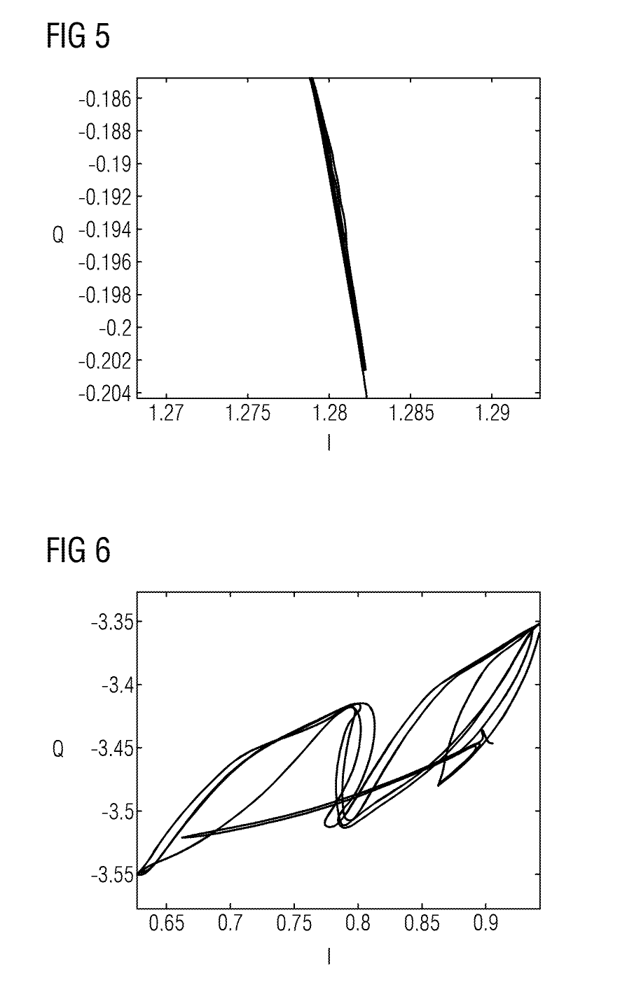

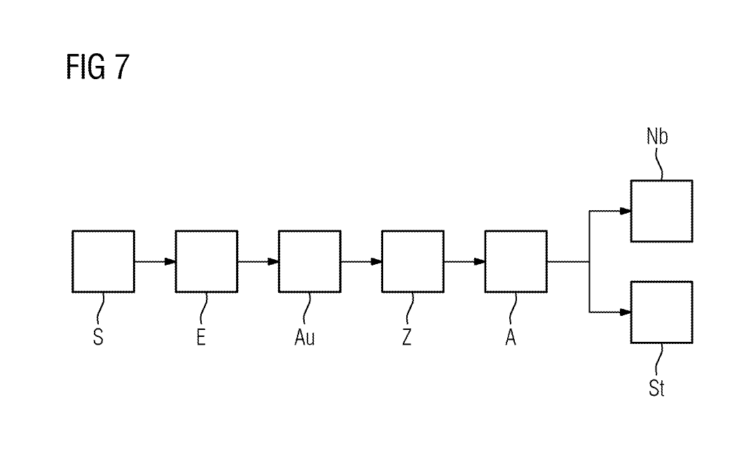

FIG. 5 shows the I and Q components of a regular motion, while FIG. 6 shows the I and Q components of two overlaid motions. In this case the Q components are each plotted on the vertical axis and the I components are each plotted on the horizontal axis. The I and Q components plotted here have been determined using the inventive method. The time curve of these I and Q components can be adjusted to model data relating to the motion of an examination region. For example, it may relate to thoracic and abdominal respiration in the case of the overlaid motions. According to an embodiment of the invention, different overlaid motions can be distinguished from one another by adjustment A of the parameters to model data.

In the case of a radar system used in continuous wave mode, the complex time-dependent transmission factor can be determined for each evaluated pair of transmit units 21 and receive units 22 in the form of the (real) I and Q component of the receive signal relative to the transmitted radar signal, as a function of the time t: I(t,j), Q(t,j) where j=1 . . . N and N is the number of antenna pairs evaluated. For other radar modes another type of signal is produced if appropriate, but generally the signal of each antenna pairing can be described as a vector U(t,j) where j=1 . . . N. The variable t may be time-continuous or else time-discrete. In the case of simple continuous wave radar, U would be a two-component vector with the elements I and Q.

In the case of multifrequency continuous wave radar, U would contain the I and Q components for each signal frequency, and thus at M signal frequencies would have 2×M components. In the case of ultra-wideband radar the elements of U would correspond to different delays (and thus intervals) between the transmitted radar signal and the received radar signal. The values of U would then describe the correlation between the transmitted radar signal and the received radar signal in the case of the respective delay. Performing An embodiment of the inventive method in multifrequency continuous wave mode is advantageous in that a variation in the frequency is synonymous with a change in the penetration depth into the body of the patient 3. As a result, motions of different examination regions at a different depth inside the body can be determined without the position of the antenna arrangement 20 changing.

The adjustment of the correlated receive signals takes place for example to a trained model of the lungs using only a few patient-specific parameters. This embodiment permits a temporally resolved evaluation of the thoracic and abdominal respiration and of the lung volume in the individual respiratory positions of the patient 3. In addition the set of parameters obtained in this way can be used to identify a particular patient 3, as the derived parameters are specific for a patient 3. This may in particular ensure that the right patient 3 is being treated on the right device. On the basis of a model of the lungs trained in this way or of an expanded whole-body model the orientation and positioning of the patient 3 can be identified, in order to avoid errors when registering the image data recorded from the patient 3. If the antenna arrangement 20 is installed in a fixed position, for example in a patient table 6, precise details of the position of the patient 3 can also be obtained using an embodiment of the inventive method, as the derived parameters depend on the position or orientation of the patient 3 relative to the antenna arrangement. Additionally it is conceivable for respiratory instructions to be given to the patient 3 with the help of an embodiment of the inventive method.

FIG. 7 shows a flow chart of an embodiment of the inventive method for determining the motion of an examination region of a patient. An embodiment of the inventive method comprises the transmission S of radar signals in the direction of an examination region, the receipt E of radar signals reflected by the examination region, and the read-out Au of receive signals from the receive units, with the receive signals corresponding to the radar signals received. Furthermore, the inventive method comprises the assignment Z of the radar signals received by the receive units 22 to the transmit units 21 which transmitted the radar signals received in each case. The assignment Z can take place by correlation of the receive signals with the control signal. The direct assignment Z of a received radar signal to a transmit unit 21 also corresponds to a spatial assignment of the received radar signal.

An embodiment of the inventive method also comprises the determination of the motion of an examination region of a patient 3. Using an embodiment of the inventive method the speed and direction of the motion of the examination region can be determined by means of the Doppler effect from a radar signal transmitted by a transmit unit 21, reflected by the examination region and then received by a receive unit 22. The determination takes place for example using the determination unit 23. An embodiment of the invention also allows the motion of a patient 3 to be determined precisely, as well as contactlessly, fast and reliably.

The determination can take place for example by adjustment A, by adjusting the digitized values of the I and Q components obtained from an I/Q demodulator 13 to retrievably stored temporal series of I and Q components which correspond to known motions of the examination region. In this case the measured I and Q components therefore assume the role of parameters which can be adjusted to model data in the form of stored I and Q components.

The determination of the motion of the examination region can in particular comprise the adjustment A of parameters derived from the correlated receive signals to retrievably stored model data, with the model data relating to the motion of the examination region. The parameters involve for example the amplitude, the mean frequency, the width of a frequency distribution or its respective Fourier-transformed values. The parameters can further involve the volume and the spatial extent, for example described by length, width and depth, of a moving examination region.

In an embodiment of the invention, the adjustment A takes place in particular to model data which relates to the change over time of the volume of the examination region. Furthermore, the adjustment can take place to model data which relates to the frequency spectrum of the motion of the examination region. If a motion of an examination region is to be determined on the basis of the respiration of the patient 3, it is advantageous to adjust the parameters both to model data for thoracic respiration as well as to model data for abdominal respiration. The model data can for example be retrievably stored on a computer 18 or a server accessible using the internet or intranet.

In an embodiment of the invention, the model data is created by training a model. Such training entails plotting signals in the form of vectors U(t,j) for a plurality of patients using the inventive antenna arrangement 20, where j=1 . . . N and N is the number of antenna pairs evaluated. In this case image data from the respective patients is recorded simultaneously with the signals U(t,j) using an imaging diagnostic device, for example a computed tomography system. Then the signals U(t,j) are subjected to a principal axis transformation in order to reduce the dimension j and thus to obtain temporally resolved vectors V(k,t) where k<j. By evaluating the vectors V(t,k) together with the synchronously recorded image data it is possible to determine the periodicity dT of the respiration and in this way to determine the temporally averaged vector

V*(k,t=t_{—}x)=[V(k,t)+V(k,t+dT)+V(k,t+2*dT)+ . . . ]

for different start times t_x.

For example, the respiratory cycle can be evaluated at five different instants t_x, as a result of which in each case a vector V*(k,t=t_x) can be determined for the different respiratory positions.

To simplify the presentation a further vector

V**(k′)=[V*(k,t=t_{—}1),V*(k,t=t_{—}2),V*(k,t=t_{—}3) . . . V*(k,t_{—}x)]

is obtained, where k′=k*x elements. In this way the temporally resolved signal U(t,j) is converted into a vector V**(k′). This vector V**(k′) must then be saved in a database with the model parameters to be determined, for example the lung volume, the shape of the lungs, etc., which can be determined from the image data. In a subsequent application a value for the respective model parameter sought can be obtained for any patients by measurement of U(t,j) and derived therefrom V**(k′) by searching in the database. Alternatively to the database, a neural network with the input variables V**(k′) and the model parameters as output variables can be trained, since in this way an automatic weighting of the elements of V** takes place.

In another embodiment of the invention the transmission S and receipt E of radar signals takes place with a scan rate of at least 10 Hz, so that the motion of the lungs of the patient 3 can be recorded. In another embodiment of the invention the transmission S and receipt E of radar signals takes place with a scan rate of at least 500 Hz, so that the motion of the heart of the patient 3 can be recorded. In both of these embodiments the radar signals transmitted from the different transmit units 21 must of course be distinguished, for example using a different frequency, a different frequency modulation or a different transmit instant. If an embodiment of the inventive antenna arrangement 20 comprises ten transmit units 21, each with an antenna, and if a scan rate of 10 Hz (or 500 Hz) is aimed for, each of the ten antennas transmits ten radar signals (or 500 radar signals) a second. The scan rate within the meaning of the present application is thus in principle independent of the number of transmit units 21.

For example, all transmit units 21 can transmit a radar signal simultaneously, each with a different frequency, in order to achieve the corresponding scan rate. Operation in continuous wave mode is then possible, so that the scan rate can be very high. Alternatively the transmit units 21 transmit radar signals one after the other, if appropriate with the same frequency. Operation is then in pulsed mode. In particular, the transmit units 21 can transmit radar signals in a fixed sequence in each cycle—i.e. the period in which each antenna transmits exactly one radar signal in pulsed operation—and which lasts a tenth of a second at a scan rate of 10 Hz. In another embodiment the inventive method is carried out in ultra-wideband mode.

In another embodiment the inventive method also comprises the control St of a medical diagnostic or therapeutic unit and/or the post-processing Nb of data obtained by a medical diagnostic or therapeutic unit, in each case using the determined motion of the examination region of the patient 3. An embodiment of the inventive method embodied in this way increases the quality of the diagnosis or treatment, for example by correcting previously recorded image data or triggering an irradiation system.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Although the invention has been illustrated and described in detail on the basis of the preferred example embodiment, the invention is not limited by the disclosed examples and other variations can be derived herefrom by the person skilled in the art, without departing from the scope of protection of the invention.

Although the invention has been illustrated and described in greater detail on the basis of the preferred exemplary embodiments, the invention is not limited by the disclosed examples and other variations can be derived herefrom by the person skilled in the art without departing from the scope of protection of the invention. In particular method steps can be performed in a different sequence from the sequences cited.

What is claimed is:

1. A method to determine motion of an examination region of a patient using a flat antenna arrangement, comprising at least one transmit unit and several receive units, the method comprising:
transmitting radar signals in a direction of the examination region using the at least one transmit unit actuated by a control signal;
receiving radar signals reflected by the examination region using the receive units;
reading-out receive signals from the receive units, the receive signals corresponding to the radar signals received;
assigning each of the respective radar signals received to the respective at least one transmit unit which transmitted the radar signals received, by correlating each of the respective the receive signals to the control signal; and
adjusting parameters derived from the correlated receive signals to retrievably stored model data which relates to the motion of the examination region.

2. The method of claim 1, wherein the adjusting takes place at least to model data which relates to the change over time of a volume of the examination region.

3. The method of claim 1, wherein the adjusting is made at least to model data which relates to a frequency spectrum of the motion of the examination region.

4. The method of claim 1, wherein the examination region relates to lungs of the patient, and wherein the transmitting and receiving of radar signals takes place at a scan rate of at least 10 Hz.

5. The method of claim 4, wherein the adjusting comprises both adjusting to model data for thoracic respiration and adjusting to model data for abdominal respiration.

6. The method of claim 1, wherein the examination region relates to a heart of the patient, and wherein the transmitting and receiving of radar signals takes place at a scan rate of at least 500 Hz.

7. The method of claim 1, wherein the model data is based on a trained model of the examination region.

8. The method of claim 1, further comprising:
post-processing image data of the examination region recorded during the receiving and transmitting using the adjusted parameters.

9. The method of claim 1, wherein the at least one transmit unit and the receive units are located in immediate vicinity of the patient, so that principally a near-field portion of the transmitted radar signals is reflected and received.

10. The method of claim 1, further comprising:
controlling a medical diagnostic or therapeutic device using the adjusted parameters.

11. A medical diagnostic or therapeutic device designed to carry out the method as claimed in of claim 1.

12. The medical diagnostic or therapeutic device of claim 11, comprising a patient table, into which the antenna arrangement is integrated.

13. The method of claim 2, wherein the adjusting is made at least to model data which relates to a frequency spectrum of the motion of the examination region.

14. The method of claim 2, wherein the examination region relates to lungs of the patient, and wherein the transmitting and receiving of radar signals takes place at a scan rate of at least 10 Hz.

15. The method of claim 2, wherein the examination region relates to a heart of the patient, and wherein the transmitting and receiving of radar signals takes place at a scan rate of at least 500 Hz.

16. The method of claim 2, further comprising:
controlling a medical diagnostic or therapeutic device using the adjusted parameters.
